(19) United States
(12) Patent Application Publication
(10) Pub. No.: US 2003/0216763 A1
(43) Pub. Date: Nov. 20, 2003

(54) Method of determining the visual axis of an eye

(76) Inventor: Anilbhai S. Patel, Arlington, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/145,447

(22) Filed: May 14, 2002

(51) Int. Cl. A61F 9/00

(52) U.S. Cl. 606/166

(57) ABSTRACT

A method of locating and marking the intersection of the visual axis and the corneal surface. With the method of the present invention, the patient fixates simultaneously at a fixation target and at a distant target laser beam for achieving coincidence of both. Fluorescein dye is placed on the corneal and fluoresces at the location where the beam intersects the cornea. This intersection point can be marked, for example, with a visible dye.

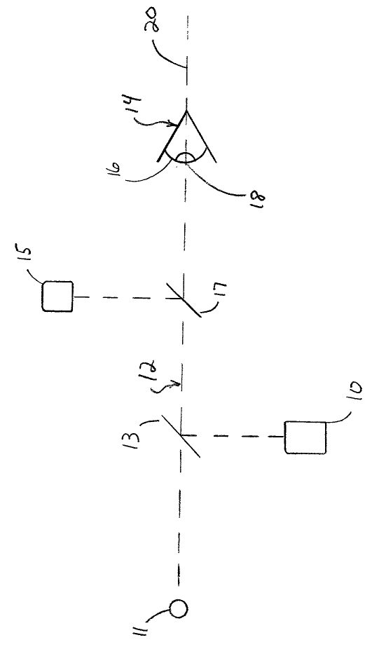

### BACKGROUND OF THE INVENTION

The present invention relates generally to ophthalmic surgery and more specifically to refractive ophthalmic surgery.

The human eye in its simplest terms functions to provide vision by transmitting light through a clear outer portion called the cornea, and focusing the image by way of a crystalline lens onto a retina. The quality of the focused image depends on many factors including the size and shape of the eye, and the transparency of the cornea and the lens.

The optical power of the eye is determined by the optical power of the cornea and the crystalline lens. In the normal, healthy eye, sharp images are formed on the retina (emmetropia). In many eyes, images are either formed in front of the retina because the eye is abnormally long (axial myopia), or formed in back of the retina because the eye is abnormally short (axial hyperopia). The cornea also may be asymmetric or toric, resulting in an uncompensated cylindrical refractive error referred to as corneal astigmatism. In addition, due to age-related reduction in lens accommodation, the eye may become presbyopic resulting in the need for a bifocal or multifocal correction device.

In the past, axial myopia, axial hyperopia and corneal astigmatism generally have been corrected by spectacles or contact lenses, but there are several refractive surgical procedures that have been investigated and used since 1949. Barraquer investigated a procedure called keratomileusis that reshaped the cornea using a microkeratome and a cryolathe. This procedure was never widely accepted by surgeons. Another procedure that had gained widespread acceptance is radial and/or transverse incisional keratotomy (RK or AK, respectively). Recently, the use of photablative lasers to reshape the surface of the cornea (photorefractive keratectomy or PRK) or for mid-stromal photoablation (Laser-Assisted In Situ Keratomileusis or LASIK) have gained widespread acceptance and commercial use in the U.S. and other countries. With the use of a photoablative laser, it is necessary to find the visual axis of the eye so that the corrective ablation pattern to be applied by the laser is correctly center on the cornea. Currently, the pupil center is chosen and used for centering the ablation. Also, wavefront measurement devices use line of sight, which goes through the center of the pupil, to compute and display various aberrations of the eye and also for deriving lower order aberrations such as sphere, cylinder and axis prescriptions for the eye. Such derived refraction prescriptions do not always agree with those prescriptions that have been measured directly in a refractive lane using a phoropter-based subjective method during which the eye looks at a target using the visual axis.

One of the reasons for this difference is the fact that the center of the pupil and the line of sight changes as the pupil dilates and is thus, neither is a stable reference. Also, the relationship between the various axes of the eye is not only complex, but thus far not easily determinable by any method. A detailed discussion is available in the following references:

1. Y. LeGrand, S. G. El Hage. Physiological Optics, Chapter 5, p. 71-74. Springer-Verlag, Berlin, Heidelberg, New York 1980.

2. Francis E. Martin. The Importance and Measurement of Angle Alpha. Br. J. Physiol Optics, 3:27-45, 1942.

The visual axis is defined as the line joining the fixation object to the first nodal point, and the second nodal point to the fovea or, if the nodal points are regarded as coincident, the line joining the fixation object to the fovea, passing through the nodal point.

The visual axis is important because it is the direction of gaze of an eye during all subjective measurements of refraction as well as for visual function of forming an image of the target of interest on the fovea. While the position of the visual axis can be ascertained by instructing the subject to fixate at a given fixation mark, its capture on its intersection with the anterior surface of the cornea is more difficult.

Accordingly, a need continues to exist for a method of locating the visual axis, preferably at its intersection with the anterior surface of the cornea. The location of such intersection can then be used for centering refractive surgical procedures, computation of wavefront measurements or any other ophthalmic diagnostic or surgical procedure.

### BRIEF SUMMARY OF THE INVENTION

The present invention improves upon prior art by providing a method of locating and marking the intersection of the visual axis and the corneal surface. With the method of the present invention, the patient fixates simultaneously at a fixation target and at a distant target laser beam for achieving coincidence of both. Fluorescein dye is placed on the corneal and fluoresces at the location where the beam intersects the cornea. This intersection point can be marked, for example, with a visible dye.

Accordingly, one objective of the present invention is to provide a method of locating the intersection of the visual axis and the anterior surface of the cornea.

Another objective of the present invention is to provide a method of locating the intersection of the visual axis and the anterior surface of the cornea that does not require locating the pupil center.

These and other advantages and objectives of the present invention will become apparent from the detailed description, drawings and claims that follow.

### BRIEF DESCRIPTION OF THE DRAWINGS

The drawing is a schematic representation of the method of the present invention.

### DETAILED DESCRIPTION OF THE INVENTION

As seen in the drawing, the method of the present invention generally includes the use of a light source 10 which projects a collimated beam of light 12 into patient eye 14 through beamsplitter 13. Light source 10 preferably is a laser projecting a beam of light in the blue wavelengths, but may also be a collimated white beam filtered by an appropriate filter to achieve a blue wavelength. Beam 12 preferably is very narrow, on the order of 0.01 mm to 0.20 mm in diameter depending upon the need for accuracy of locating the visual axis and the sensitivity of the system to capture the fluorescein excitation location. By providing the patient with fixation target 11 and having patient eye 14 fixate on target 11 (through beamsplitters 13 and 17) so that fixation target 11 and beam 12 are coincident, the visual axis of eye 14 will center on beam 12.

During use, a fluorescein dye, or other suitable dye, is placed on the cornea 16 and will fluoresces at location 18 where beam 12 intersects cornea 16. Intersection 18 corresponds to the location of visual axis 20 of eye 14 at cornea 16. Once located by the fluorescence of the dye, intersection 18 may be visibly marked, such as by a visual marker or captured by camera 15 along with other registration markings such as the limbus and/or scleral and/or pupillary vessels, and beam 12 may be discontinued. Intersection 18 may now be used to center any refractive procedure on cornea 16. If camera 15 has been used and intersection 18 along with other registration markings have been captured, intersection 18 is available for future use.

This description is given for purposes of illustration and explanation. It will be apparent to those skilled in the relevant art that modifications may be made to the invention as herein described without departing from its scope or spirit. For example, the invention as may use other excitation wavelengths appropriately selected for other corresponding dyes which could provide emission of visible light or even light invisible to humans but sensed by an appropriate camera or other optical sensing device.

I claim:

1. A method of locating the intersection of a visual axis of an eye and the cornea of the eye, the method comprising:
i) providing a fixation target;
i) projecting a collimated light beam into an eye;
ii) focusing the eye such that the collimated light beam and the fixation target coincide;
iii) placing a fluorescing dye on a cornea of the eye; and
iv) locating a point where the collimated light beam intersects the cornea by observing the location on the cornea where the dye fluoresces.

2. The method of claim 1 further comprising the step of placing a visible mark at the point where the light beam intersects the cornea.

3. The method of claim 1 further comprising the step of capturing a video image of the point where the light beam intersects the cornea.

4. The method of claim 1 wherein the collimated light beam is a visible beam of light in the blue wavelengths.

5. A method of locating the intersection of a visual axis of an eye and the cornea of the eye, the method comprising:
i) providing a fixation target;
i) projecting a collimated light beam in the blue wavelengths into an eye;
ii) focusing the eye such that the collimated light beam and the fixation target coincide;
iii) placing a sodium fluorescein dye on a cornea of the eye;
iv) locating a point where the collimated light beam intersects the cornea by observing the location on the cornea where the dye fluoresces;
v) placing a visible mark at the point where the light beam intersects the cornea.

6. The method of claim 5 further comprising the step of capturing a video image of the point where the light beam intersects the cornea.
